(19) United States
(12) Patent Application Publication
(10) Pub. No.: US 2009/0043381 A1
(43) Pub. Date: Feb. 12, 2009

(54) Atrioventricular valve annulus repair systems and methods including retro-chordal anchors

(76) Inventors: John A. Macoviak, La Jolla, CA (US); Robert T. Chang, Belmont, CA (US); Alden Harken, Walnut Creek, CA (US); Timothy R. Machold, Moss Beach, CA (US); David A. Rahdert, San Francisco, CA (US)

Correspondence Address: RYAN KROMHOLZ & MANION, S.C., POST OFFICE BOX 26618, MILWAUKEE, WI, 53226, US

(21) Appl. No.: 11/664,545

(22) PCT Filed: Oct. 5, 2005
(86) PCT No.: PCT/US2005/035750

§ 371 Date: Mar. 25, 2008

Related U.S. Application Data
(60) Provisional application No. 60/616,139, filed on Oct. 5, 2004.

(51) Int. Cl. A61F 2/24; A61B 17/04

(52) U.S. Cl. 623/2.36; 606/232; 623/2.38

(57) ABSTRACT

Methods, devices and systems are disclosed to treat atrioventricular valve regurgitation accessed through the vasculature, and by standard and minimally invasive surgical techniques. Isolated leaflet fixation and annulus treatment systems are developed.

### RELATED APPLICATIONS

This application claims the benefit of U.S. Provisional Patent Application Ser. No. 60/616,139, filed Oct. 5, 2004, and entitled “Atrioventricular Valve Annulus Infra-Retro-Leaflet and Suspension Systems and Methods.”

### FIELD OF THE INVENTION

The field of repairing human heart valves has evolved from standard surgical to less invasive surgical to endovascular methods and devices. The current invention has technologies that may be applied by all three methods. Special application by the least invasive endovascular means are fully developed in the disclosure of the methods, devices and systems of this invention. Leaflet fixation by anchoring and buttressing means, leaflet suspension, annulus shortening and stabilization capabilities enable treatment of most forms of atrioventricular valve regurgitation.

### BACKGROUND OF THE INVENTION

There are two atrioventricular heart valves, the mitral and tricuspid heart valves. Their function and form are similar and each is amenable to similar therapies and subject to similar pathologies. Mitral valve regurgitation will be used as the primary example in this disclosure. However the same principles, device, systems and methods may be applied to the tricuspid valve.

Anatomically the mitral valve has two leaflets named the anterior and posterior leaflets. Compared to the posterior leaflet, the anterior leaflet has a larger surface area and larger length from its insertion at the mitral annulus on the heart wall to its tip where the chordae tendineae begin. The latter connects the leaflet to the papillary muscles of the left ventricle by thin linear fibrous strands that are approximately 1 to 3 cm long to the papillary muscles of the left ventricle. The anterior leaflet inserts on approximately one third of the circumference of the mitral annulus which is fibrous while the posterior leaflet inserts on the remaining two thirds of the annulus which is fibromuscular.

Functionally in ventricular diastole the ventricular muscle relaxes and the heart fills with blood. In the early phase of diastole the mitral valve is open as the ventricle is its most empty. The leaflets have fallen into the ventricle. In addition the atrium may contract and eject blood through the mitral valve opening it further. The mitral valve begins to close as the ventricle fills with blood and the blood beneath the mitral leaflets forces them upward and inward relative to the central mitral valve orifice. When the ventricle begins to contract in systole the leaflets become further advanced upward and inward and together by the higher blood pressure in the ventricle if the mitral valve is not regurgitating blood. The closing of the mitral valve allows more effective emptying of blood from the left ventricle into the aorta so that it does not leak retrograde into the left atrium during ventricular systole.

Mitral regurgitation like tricuspid valve regurgitation is an abnormal retrograde flow of blood through a heart valve. It occurs during ventricular systole and late diastole. It may be as a result of annulus dilation when it is called functional regurgitation. The etiology of functional mitral regurgitation is un-remedial congestive heart failure that causes significant dilation of the left ventricle and mitral annulus. When substantial annulus dilation occurs the mitral leaflets become unable to completely coapt or approximate in systole. The leaflets are placed under tension at the annulus which has separated anterior from posterior leaflets due to annulus dilation. Also, further tension on each leaflet occurs at the level of the chords and papillary muscles that are pulled down and out by the wall of the dilated left ventricle. Differently, when the cause of mitral regurgitation is structural or organic it may occur as a result of a ruptured chord, or leaflet prolapse, or an infectious necrotizing process that can involve any component of the valve. These lesions cause failure of the leaflets to remain competent during systole.

Generally today if mitral regurgitation is significant and the patient is well enough to tolerate surgery it is treated surgically using cardiopulmonary bypass techniques. If there is poor ventricular function though, surgery can extract a high morbidity and mortality.

There have been prior attempts to treat mitral regurgitation by means of endovascular technologies through the vasculature into the beating mechanically unsupported heart. Several techniques have come under evaluation for use in man; however, none of them has gained approval for use in man to date. These other techniques differ significantly from the current invention.

Ideally endovascular repair techniques would avoid the complications of the higher risk surgical techniques. In pursuit thereof, the following invention offers novel endovascular technologies to repair mitral regurgitation on the beating, mechanically unsupported heart, due to functional dilation of the annulus or due to structural organic lesions of the mitral valve leaflet, chords or papillary muscles. These technologies may also be applied by any surgical approach as well.

### SUMMARY OF THE INVENTION

All mitral valve device positions are given relative to the central normal antegrade flow of blood through the mitral valve orifice. The term central refers to blood flow through the center of the valve orifice between the two leaflets. The term upstream refers to going in the opposite direction of the normal blood flow. The term downstream refers to blood flow in the normal direction past the point of reference.

All classes of these devices may be constructed of man-made shape-memory alloys, elastic alloys, polymer plastics, and naturally occurring metals or other substances.

These devices, systems and methods may be applied by any established beating heart or open heart direct surgical approach using relatively short catheters or tools, e.g., less than approximately 80 cm in length, or with standard surgical tools. Or these devices, systems and methods may be applied with longer catheters, e.g., greater than approximately 80 cm in length, for endovascular applications.

One aspect of the invention provides a core class of repair methods, devices and systems, which relate to retro-chordae tendineae-anchors. The anchors fix at least part of at least one mitral leaflet into a preferred position. Retro-chordae tendineae-anchors may be bridged or bonded to an opposing-atrial-related-anchor located in or near the right atrium, vena cava or the left atrium or great coronary vein.

Retro-chordae tendineae-anchors may be expandable or inflatable multidimensional or linear tubular or solid linear devices in form. They are catheter deliverable or surgically usable devices placed behind the valve chordae tendineae.

Retro-chordae tendineae-anchors are designed for spreading anchoring forces across as many chords as is feasible to prevent retro-chordae tendineae rupture while allowing a leaflet to be pulled by an opposing anchor upstream and centrally. A retro-chordae tendineae-anchor has at least one bridge or tissue-to-tissue bonded attachment extending to or from it to enable a pulling, bridging or bonding function with another opposing anchor located elsewhere in the heart.

Retro-chordae tendineae-anchors may be stabilized by hooking, stapling, gluing or by other means attaching to a chord or other nearby structure to prevent migration of the anchor from an implanted position.

The collaborative devices necessary for retro-chordal devices to function include great coronary vein anchors, suspension-scaffold anchors, right atrial septal anchors, vena cava anchors and various anchor to anchor bridging and bonding elements.

Both mitral valve leaflets and all three leaflets of the tricuspid valve may also be amenable to these therapies.

Another aspect of the invention provides repair methods, devices and systems comprising an infra-leaflet-buttress. The infra-leaflet-buttress is a device that, depending upon the surgical application, may be fixed in size or may be expandable, inflatable and multidimensional or linear and fixed in size for a catheter deliverable device. In use, this device may be placed beneath a valve leaflet in a retro-chordae tendineae and infra-leaflet position. Infra-leaflet-buttresses are used for stretching out or filling up and out a leaflet by a mass effect to physically displace a leaflet from a retracted more open position into instead a more closed upstream and centrally located position. An infra-leaflet-buttress may be a self-contained device without attachments or it may have attachment mechanisms only for securing it to immediately surrounding contiguous structures.

### BRIEF DESCRIPTION OF THE DRAWINGS

FIG. 1a is a sagittal view of the left heart through the middle of the anterior and posterior leaflets of the mitral valve. This view is the heart in systole when the leaflets should be closed but cannot meet fully because of a dilated heart and annulus condition causing functional mitral regurgitation. The same heart is shown in diastole in FIG. 1b.

FIG. 2a is a sagittal view of the left heart through the middle of the anterior and posterior leaflets of the mitral valve. This heart has prolapse of the posterior leaflet of the mitral valve with mitral regurgitation during systole in FIG. 2a. The same heart is shown in diastole in FIG. 2b.

FIG. 3a is a sagittal view of the left heart through the middle of the anterior and posterior leaflets of the mitral valve. This view is the heart in systole with a ruptured chordae tendineae condition of the middle scallop of the posterior leaflet of the mitral valve leading to a flail leaflet causing organic or structural mitral regurgitation. The same heart is shown in diastole in FIG. 3b.

FIG. 4a is a sagittal view of the left heart through the middle of an anterior and a flail posterior leaflet of the mitral valve. Shown is an anterior leaflet retro-chordae tendineae-anchor bonded to a posterior atrial wall anchor located inside the great coronary vein. The anterior leaflet is fixed into a more than normally occurring upstream and central or posterior position for a given heart. This system may not be able to treat mitral regurgitation due to a dilated annulus as the anterior leaflet may not reach the posterior atrial wall, see FIG. 5. Bonding may however treat a leaflet or an opposing leaflet having a prolapse or as in this case a ruptured chord condition of either the anterior or posterior valve leaflet. In this view the posterior mitral leaflet has become totally roofed by the anterior mitral leaflet and the posterior atrial wall. The posterior mitral annulus has also been moved more anteriorly.

FIG. 4b is a transverse section through the top of the atrioventricular heart valves shown in the systolic phase of the cardiac cycle of the same heart as in FIG. 4a. In this view the posterior mitral valve leaflet is seen to coapt well with the anterior leaflet.

FIG. 4c is a transverse section through the top of the atrioventricular heart valves shown in the diastolic phase of the cardiac cycle of the same heart as in FIG. 4a. In this view the posterior mitral valve leaflet is seen to open well away from the anterior leaflet.

FIG. 5a is a sagittal view of the left heart through the middle of an anterior and a flail posterior leaflet of the mitral valve. Shown is an anterior leaflet retro-chordae tendineae-anchor bridged but not bonded to a posterior atrial wall anchor located inside the great coronary vein. The anterior leaflet is fixed into a more than occurring upstream and central or posterior position. This system may also treat mitral regurgitation due to a dilated annulus, or of a treated leaflet or an opposing leaflet having a prolapse or as in this case a ruptured chord condition of either the anterior or posterior valve leaflet. In this view the posterior mitral leaflet has become totally roofed by the anterior mitral leaflet and the posterior atrial wall. The posterior mitral annulus has also been moved more anteriorly.

FIG. 5b is a transverse section through the top of the atrioventricular heart valves shown in the systolic phase of the cardiac cycle of the same heart as in FIG. 5a. In this view the posterior mitral valve leaflet is seen to coapt well with the anterior leaflet.

FIG. 5c is a transverse section through the top of the atrioventricular heart valves shown in the diastolic phase of the cardiac cycle of the same heart as in FIG. 5a. In this view the posterior mitral valve leaflet is seen to open well with away from the anterior leaflet.

FIG. 6a is a sagittal view of the left heart through the middle of an anterior and a flail posterior leaflet of the mitral valve. A left atrial suspension-scaffold is seated with a strut in each mitral commissure. A bridging element suspends a retro-chordae tendineae-anchor from behind the anterior leaflet chords. The posterior mitral leaflet is partially roofed and coapts with the anterior leaflet during systole.

FIG. 6b is a transverse section through the top of the atrioventricular heart valves shown in the systolic phase of the cardiac cycle of the same heart as in FIG. 5a. In this view the posterior mitral valve leaflet is seen to coapt well with the anterior leaflet.

FIG. 6c is a transverse section through the top of the atrioventricular heart valves shown in the diastolic phase of the cardiac cycle of the same heart as in FIG. 5a. In this view the posterior mitral valve leaflet is seen to open well away from the anterior leaflet.

FIGS. 7a-c are similar to FIGS. 6a-c except that there are two separate bridging elements each individually suspending a retro-chordae tendineae-anchor from behind each the anterior and the posterior mitral leaflets proximate their middle scallops. This repair system may treat mitral regurgitation potentially from all etiologies.

FIG. 8a shows an infra-leaflet-buttress applied to a posterior mitral leaflet in a heart with a dilated mitral annulus in systole.

FIG. 8b shows an infra-leaflet-buttress applied to a posterior mitral leaflet with a ruptured chordae tendineae in systole.

FIG. 8c shows an infra-leaflet-buttress applied to a floppy posterior mitral leaflet with prolapse in systole.

FIGS. 9a and 9b show a posterior mitral leaflet retro-chordae tendineae-anchor with a bridging element to a tubular great coronary vein anchor that may be effective for treating all forms of mitral regurgitation.

FIG. 10a shows a superior vena cava anchor with a trans-septal bridging element to an anterior mitral leaflet retro-chordae tendineae-anchor that may be effective in treating all forms of mitral regurgitation.

FIG. 10b is the same as 10a but the opposing more anterior anchor is located on the atrial septum.

FIG. 11a is a sagittal view through the middle of the anterior and posterior leaflets of the mitral valve and FIG. 11b is a transverse section through the top of the atrioventricular heart valves of the left heart. Shown is a retro-chordae tendineae-anchor of the anterior mitral leaflet bonded to a great coronary vein tubular anchor. Bonding is accomplished by magnets that are located in each anchor.

FIG. 12a is a sagittal view of the heart through the left ventricular outflow tract anterior to the anterior-leaflet of the mitral valve and its chordae tendineae. A method of delivery of a T shaped great coronary vein anchor through the vasculature and through the aortic valve into the left atrium and then into the great coronary vein is shown. Other methods of delivery can be used, for example, as disclosed in U.S. patent application Ser. No. 11/089,939 filed Mar. 25, 2005, and entitled Devices, Systems, and Methods for Reshaping a Heart Valve Annulus,” which is incorporated herein by reference.

FIG. 12b is a continuation of the FIG. 12a delivery system showing deployment of a bridging element from the great coronary vein anchor back through the now deployed anterior mitral valve leaflet retro-chordae tendineae-anchor.

FIG. 12c is a continuation of the FIG. 12b delivery system showing final seating of the anchor system on the bridging element and the catheter delivery system removed.

FIG. 12d is a sagittal view of the heart through the left ventricular outflow tract anterior to the anterior leaflet of the mitral valve and its chordae tendineae. A delivery catheter is shown bringing a bridge and proximal anchor to link with a stent in the great coronary vein.

FIG. 12e is a continuation of the FIG. 12d delivery system now showing full attachment of the jaws of the bridging element onto the strut of the great coronary vein stent.

FIG. 12f is a continuation of the FIG. 12e delivery system showing final seating of the anchor-bridge-anchor system.

FIG. 13a is a schematic drawing of the initial stage application of an in the heart delivery catheter, bridging element and proximal anchor and control elements.

FIG. 13b is a continuation of the next stage application of FIG. 13b where the outermost delivery sheath has been removed allowing the compressed distal jaws and proximal anchor to open and expand.

FIG. 13c is the next stage application of FIG. 13b where the anchor-bridge-anchor system is fully deployed and locked with the proximal control elements removed.

FIG. 14 is a schematic of a wire form central bridging element bridge component used to link two anchors described in the invention.

FIG. 15 is a schematic of three of the several possible embodiments claimed of great coronary vein large cell stent-like anchors.

FIG. 16 is a schematic showing a cross-section of a great coronary vein within which is a four-longitudinal strut anchor and locked-on jaws of a bridging element.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

### I. Repair Systems

A. Generally

In retro-chordal anchor repair, both functional dilated annulus induced mitral regurgitation and organic, also called structural, mitral regurgitation may be treated. The latter refers to cases where there are missing destroyed leaflet segments, ruptured chords, flail or prolapse conditions of either or both leaflets. These repairs function by virtue of creating an upward or upstream and forward or centrally directed parachute-like reformation and fixation of a leaflet. These system attributes further consolidate the function of a treated leaflet by spreading a newly created tension up and down all the intact chords and uniformly drawing that tension across the leaflet.

A retro-chordae tendineae-anchor may be delivered with a biocompatible covering. This covering when the device is expanded may serve to obstruct gaps in a leaflet tissue or along a coaptation line to further serve in causing a parachute-like formation to the leaflet. It also creates an adjunctive gap sealing mechanism to prevent the regurgitant flow of blood. A retro-chordae tendineae-anchor may or may not have a leaflet filling up and filling out function.

A retro-chordae tendineae-anchor may be a slender device having a leaflet tensioning function that is not capable of filling-out a leaflet.

Typically, in one preferred embodiment, at least a middle scallop of a leaflet is held in a same fixed degree of closed position up to being maximally stretched upward and inward, being as fixed closed during diastole as it is in systole. Its open position however may vary if the leaflet is less than fully fixed centrally and there is at least some lateral laxity throughout the leaflet in diastole. In leaflets where only a narrow radial of a single leaflet is fixed to some degree, the leaflet tissue on either side of it if lax enough may open or close in response to hemodynamic pressure gradients. Otherwise a fixed lateral mitral orifice is created. The opposing non-fixed leaflet may function to open and close and may fill the gap at the lateral aspect of the centrally fixed but laterally variably mobile leaflet tissue.

To further expound, in this embodiment, other segments of a valve leaflet that are not tensed or fixed in all of the heart cycles will allow other areas of a treated mitral leaflet to open enough to allow a significant degree of antegrade forward blood flow during ventricular diastole.

Upward and central primary leaflet fixation repairs by retro-chordae tendineae-anchored fixation mechanisms may also have salutary effects on structural etiologies of mitral regurgitation primarily by virtue of leaflet stabilization. This may apply to flail or prolapsing segments of a leaflet relative to the remaining intact chords, papillary muscles and leaflet structure. In the case of structural absence of destroyed areas of leaflet tissue a biocompatible covered device as described above may fill in gaps as well as function through leaflet stabilization. In essence the leaflet stabilization mechanism is one of near consolidation of all otherwise unsupported dysfunctional valve leaflet, chordae tendineae and papillary muscle components.

In retro-chordae tendineae-anchor repair systems there is a component leaflet tension created and a separate component tension that is directed toward a posterior anchor in the great coronary vein or to another retro-chordae tendineae-anchor or up and or across a suspension-scaffold anchor bridge, or across an atrial septum to a right atrial or vena cava anchor.

In one case of an anterior leaflet being anchored to a great coronary vein anchor and bridged under some tension the anterior leaflet will come to be overlying or roofing a posterior leaflet to some extent. Anterior leaflet over posterior leaflet roofing if created provides an enhanced coaptation surface area between the two leaflets. A coaptation surface area that is an overlying roof is constituted of the anterior leaflet and chords. This will be abutted by the posterior leaflet upstream surface which abuts the downstream surface of the anterior leaflet and its chords.

The aforementioned coaptation functions of the two mitral leaflets allow upstream surfaces of a valve leaflet opposing a retro-chordae tendineae-anchored leaflet to close up against the latter leaflet that has an enhanced fixed surface area due to being bridged or bonded to an opposing anchor. The surface-area-spreading-function of the retro-chordal fixation devices applied to directly treat a leaflet also creates in itself a direct fixed barrier to retrograde or regurgitant blood flow that becomes more easily regulated by a more mobile opposing leaflet.

Posterior anchors in the heart anatomy are the great coronary vein and the posterior mitral leaflet. Opposing anterior anchors may be an anterior mitral leaflet, a suspension-scaffold in the left atrium, a trans-septal right atrial or vena cava anchor.

For a posterior leaflet retro-chordae tendineae-anchor an opposing anchor could be a right atrial-related anchor, a vena cava anchor, a posterior great coronary vein anchor, or a suspension-scaffold located above the annulus.

In the case of a posterior mitral leaflet retro-chordae tendineae-anchor, the flow of blood through the mitral valve relies upon the opening and closing of the freely mobile anterior mitral leaflet against the at least partially anchored and tensed chords and or buttressed leaflet proper.

In a separately delivered device design a fluid-filled or another substance-filled balloon-like retro-chordae tendineae-anchor is ovoid or is somewhat less structured and less than tensely filled and is amorphous or malleable. In either case it can assume a shape into which it is forced and is easily compressible. It is loaded into a catheter, compressed and then expanded when placed into position.

In the case of the anterior leaflet of the mitral valve, a retro-chordae tendineae-anchor requires construction so that in its final form it is fixed behind the anterior surface of the chords and is prevented from moving posteriorly through the chords. A retro-chordae tendineae-anchor cannot be allowed to expand either fully toward the ventricular septum anteriorly or inferiorly. It must be fixed and formed into a final position so as to not obstruct or migrate into the left ventricular outflow tract which is located anterior to the anterior mitral valve annulus. It also may not extend inferiorly below the lower limit of the chords as this is an inlet to the left ventricular outflow tract. It must not extend into the range of the papillary muscles. This is primarily in order to allow unobstructed blood flow through the mitral orifice and on into the left ventricular outflow tract in route to the aorta.

In certain embodiments by under-sizing or over-sizing a retro-chordae tendineae-anchor relative to the space into which it will be confined, and by it having a compressible outer form, a retro-chordae tendineae-anchor may conform to nearly fully fill or fully fill the space into which it is launched and confined.

A fully formed planar retro-chordae tendineae-anchor or a three dimensionally outwardly pressing embodiment may be disposed in a filled-out, non-planar geometric shape. These embodiments may be for example ovoid, spherical, single or multi-lobed prostheses. When a more fully formed and shaped three-dimensional retro-chordae tendineae-anchor is expanded in its confined space behind the chords of the valve to be treated, it may in preferred embodiments be compressible. That is, it may be over-sized and thus formable in its final shaped by the confines of the space into which it is placed. In order to be compressible it may be formed as a memory shaped alloy mesh or latticed or spiraled figure or as a balloon-like structure.

Advantages to these latter embodiments may include simpler delivery, easier fixation, and adjustable precision of inflation for optimal leaflet augmentation.

In some preferred embodiments for certain types of valve lesions retro-chordae tendineae-anchors may be soft and not rigid, to allow more natural leaflet coaptation lines to better seal.

B. Blood Flow and Leaflet Repair Systems

A retro-chordae tendineae-anchor to opposing-atrial-anchor repair increase a treated leaflet surface area that becomes spread across an orifice of a valve annulus. The fixed leaflet component in essence decreases an effective valve orifice area against both regurgitant flow and forward mitral blood flow. An effective mitral valve orifice area is measured normally in diastole with forward blood flow. It is an assessment of the opening of the mitral valve in ventricular diastole as blood flows maximally from the atrium to the ventricle. This may be measured by standard echocardiographic or cardiac catheterization techniques. In the normal situation a mitral valve orifice may measure 4 to 5 cm square. In mitral stenosis it measures <2.5 cm square. In a dilated annulus situation it may measure typically 8 cm square.

In the latter group of dilated annulus hearts, by fixing the anterior leaflet into a fully closed position, if a mitral orifice area drops by approximately 50% to approximately 4 cm square in cases of dilated annulus that is adequate for essentially normal forward blood flow.

Retro-chordae tendineae-anchor technology should cause the least amount of impediment to mitral valve inflow as is possible. That is, there should not be significant mitral stenosis created as a trade-off to treating mitral regurgitation.

Blood flow through a mitral orifice with a retro-chordae tendineae-anchor repair is determined by the degree of closed leaflet fixed positioning such devices apply to a leaflet. Then the flow around the leaflet fixation configuration at its sides, and at its leading edge if a bridge and not a bond were used, in a retro-chordae tendineae-anchor to opposing anchor configuration is what determines valve forward flow. If a single leaflet is treated then the other leaflet may open and close more effectively, assuming it is normal, up against the treated leaflet.

The flow of blood through the mitral valve relies in some part upon the opening and closing of an untreated valve leaflet opposing a treated leaflet. The untreated leaflet may be a freely mobile. It possibly may be a roofed anterior or posterior mitral leaflet. It may be closing against a roof created by tissue of an atrium extended by a pulled forward anchor in a great coronary vein that is bridged or bonded to an opposing anterior or posterior mitral leaflet retro-chordae tendineae-anchor. Additionally there may be flow in open spaces, if any are allowed around a treated leaflet after tension in the anchored or buttressed leaflet is applied. This flow may occur during ventricular systole or diastole, lateral to the treated leaflet on either side. If a treated mitral leaflet is less than fully closed at its leading central edge, that is it is bridged but not fully bonded to an opposing anchor in its fixed position then flow along the leading coaptation edge of the middle scallop, then flow through center of the mitral valve orifice to some degree would also be allowed to occur during ventricular diastole.

If a single leaflet is treated then other leaflet may open and close more effectively up against the treated leaflet. Areas of the leaflet not tensed by the repair may open and or close in cycle with hemodynamic changes in the ventricle.

### II. Anchor Systems and Combinations

There are four atrial related core anchor systems that may work in opposing positions in conjunction with retro-chordae tendineae-anchors. There are also separate combinations of these anchors that may independently work together with great coronary vein anchors and without retro-chordae tendineae-anchors to perform annulus based mitral repairs.

A. Great Coronary Vein Anchor Systems

First in the group of opposing atrial related anchors is a great coronary vein anchor. These are a group of expandable multidimensional or linear tubular devices, which may also be delivered of fixed construction. They are placed usually through the coronary sinus or into a posterior atrial wall position located within a great coronary vein. These anchors have at least one bridge extending to an anchor located more anteriorly which may include to a retro-chordae tendineae-anchor for suspending an anterior or posterior mitral valve leaflet, or to a suspension-scaffold, or to a trans-septal right atrial or vena cava anchor.

An anchor located in a posterior atrial wall great coronary vein may include but not be limited to a stent or a tubule or a solid rod that may be of any configuration that exerts a longitudinal force within and along a greater part of the length of the great coronary vein. The great coronary vein anchor can comprise a stent-like structure made of a self-expanding shape memory alloy or a malleable balloon expandable alloy.

The intended use of this anchor is to hold in tension a bridging element between the posterior and anterior anchoring regions and thereby apply a stabilizing force for an anterior leaflet retro-chordae tendineae-anchor. The stabilization force applied by the great coronary vein anchor can exist without causing movement of the atrial wall, or without causing movement of the posterior mitral annulus and of the ventricular wall to which that portion of atrial wall is attached. The stabilizing force applied by the great coronary vein anchor can also exist without shortening the major or minor axes of the valve annulus itself. The great coronary vein anchor leads to an attachment of the anterior leaflet to the great coronary vein without necessarily imposing a forward movement of the great coronary vein itself. The anchor establishes a fixed length relationship between the anterior leaflet and the great coronary vein that is stabilizing and not necessarily shortening in its effect.

B. Suspension-Scaffold Anchor Systems

Second in the group of retro-chordae tendineae-anchor opposing atrial related anchors is the suspension-scaffold atrial anchor class. This is an expandable scaffold type of device that rests on a mitral annulus and or on an atrial wall. It has at least one bridge extending from it to a retro-chordae tendineae-anchor for suspending a valve leaflet or to a great coronary vein anchor.

A suspension-scaffold may be secured at a point in or near a left atrium wall and annulus with extension of its struts across a mitral annulus into a left ventricle. Commonly a suspension-scaffold in certain embodiments would be seated with its downstream seating points being formed as pins or trans-annular struts, at or near an annulus and its most upstream seating being a curvilinear strut of a scaffold lying along a dome of a left atrium.

In a retro-chordae tendineae-anchor to atrial suspension-scaffold bridged anchor repair system a retro-chordae tendineae-anchor becomes suspended from a suspension-scaffold that is based on an annulus and or on an atrial wall endocardium. A suspension-scaffold is self-retaining by virtue of expansion against an annulus and or atrial wall. A suspension-scaffold in combination with a bridge that attaches to at least one retro-chordae tendineae-anchor moves an anchored mitral leaflet into a preferred upstream and central position. Either one or both leaflets may have retro-chordae tendineae-anchors that may be suspended from at least one suspension-scaffold.

In one embodiment a suspension-scaffold may insert its downstream seating points as pins or curve struts that cross an annulus while instead its upper most curvilinear portion lie along an atrial wall on or proximate a mitral annulus. From this position if the curvilinear portion of the scaffold is oriented anteriorly it then lies in the most part on or near the anterior annulus. From this position if the curvilinear portion of a scaffold is oriented posteriorly it then lies in the most part on or near the posterior annulus.

Also a great coronary vein anchor may anchor to an anterior annulus related suspension-scaffold through a bridging element.

C. Right Atrial Septal Anchor Systems

Third in the group of retro-chordae tendineae-anchors opposing atrial related anchors are right atrial related anchors that may interact with left atrial related anchors. These devices may be implanted on a right atrial septum usually by a trans-venous endovascular approach.

A bridge must be extended to or from a right atrial related anchor through a fossa ovalis of an atrial septum to or from a left atrial based retro-chordae tendineae-anchor or a great coronary vein anchor.

Septal anchors may for example be modeled after a standard septal occluder device used for treating patent foramen ovale. This typically is a meshwork of nitinol. Or it may be a T-shaped tubular rod that may be an alloy or a polymer in another embodiment.

Septal anchors must be non-obstructive to vena cava and right heart blood flow. When tension is placed upon a septal anchor displacement of the septum must not be sufficient enough to impinge and distort the aortic or tricuspid or mitral heart valves or the pulmonary venous drainage.

D. Vena Cava Anchor Systems

Fourth in the group of anchors opposing the retro-chordae tendineae-anchors and great coronary vein atrial related anchors is the class of vena cava right atrial related anchors. These devices may be implanted in the superior and or inferior vena cava.

A bridge must be extended to or from a right atrial related anchor through a fossa ovalis of an atrial septum to or from a left atrial based retro-chordae tendineae-anchor or a great coronary vein anchor.

A vena cava anchor in one embodiment may be formed around a stent-like foundation with mechanisms to allow attachment of a bridge to a retro-chordae tendineae-anchor. Expansion of a stent in a vena serves to anchor the stent therein. An integral channel for a bridging element that may pass through and lock onto the stent may be applied.

Vena cava anchors must be non-obstructive to vena cava, right heart and near by organ blood flow such as from the liver.

There is generally at least up to 5 cm of inferior vena cava below the entry of the inferior vena cava into the right atrium and above the liver that may safely hold a stent without compromise of blood flows.

E. Annulus Based and Combination Anchor Repair Systems

Combination repairs derive from the opposing anchor systems described above.

Collaborative double retro-chordae tendineae-anchor arrangement options include bridging together two separate anterior and posterior mitral leaflet retro-chordae tendineae-anchors. Or two anchored leaflets may be directly bridged independently or in a conjoined bridge to a suspension-scaffold or to a great coronary vein anchor or to a right atrial or a vena cava anchor system.

One combination is to use at least one suspension-scaffold to anchor a retro-chordae tendineae-anchor and with another bridging element to anchor a great coronary vein anchor.

Combinations of devices into varieties of kits generically called anchor-bridge-anchor kits for transvascular delivery and implant systems to treat atrioventricular valve regurgitation unique to this invention are described as follows.

A kit will have an outer containment-catheter for transvascular delivery with a hollow lumen and with proximal and distal apertures.

A containment-catheter will have near its distal end keeps within it in compressed form jaws at a distal end of a central bridging element and keeps within it in compressed form a proximal anchor mounted on sleeve cylinder within the lumen of which is a central bridging element all of which is in the lumen of an outer catheter and assembly.

Further the same containment catheter more proximally contains within its lumen a slotted pushing catheter with hollow lumen having proximal and distal apertures and within the latter lumen is located a pulling-catheter that may have a hollow lumen that contains a controllable distally acting grasping and releasing mechanism to grasp and release a central bridging element.

An integrally formed central bridging element that will have jaws on a distal terminus and at least one one-way chevron brace on its body and at east one proximal loop for engagement with a proximal pulling instrument.

A sliding bridge-cylinder-like outer sleeve will be located just inside the outer containment-catheter lumen that is mounted just outside the central bridging element and keeps within it in compression at least one chevron brace.

A sleeve will contain a central bridging element the former of which is hollow and has a proximal and a distal aperture.

An intra-containment-catheter pulling element is reversibly attachable to a loop formed at a proximal terminus of a central bridging element.

A distal end of a sleeve will have male type seating sites corresponding to female type seating points on the distal jaws of the central bridging element.

A slotted hollow open-ended pushing catheter engages its male type distal aspect with a female type proximal aspect of a sleeve the latter being mounted over a central bridging element.

The pushing catheter is slotted respectively to allow a wing of a chevron to advance proximally when a central bridging element is pulled against a pushing catheter holding a sleeve.

The outer containing-catheter may have near its distal tip a magnetic or ferromagnetic material.

### III. Bridging and Bonding Systems

The separate class of devices that connects anchors together and prevents them from migration is the class of bridging and bonding devices.

Bridging and bonding systems interact directly with the anchor to anchor systems described herein for the treatment of functional and organic or structural atrioventricular valve regurgitation.

In certain embodiments inflatable, expandable or non-expandable embodiments of anchors and bridging devices may be used. Anchors and bridges may be delivered as integrally constructed devices or as separately delivered and applied devices. Non-expandable devices may be especially useful in some endovascular and some surgical cases. In certain embodiments catheter delivered devices may also be linear and non-expandable.

Bridging element construction materials may include shape memory alloys, synthetic or natural polymers, elastic materials, inelastic materials, mating magnets, or a magnet and a ferromagnetic mate, wires, cables, staples, screws, snap-ins, cinching or locking, bridging or linking mechanisms, or of any other known connector devices of any variety.

Catheter jaws or stapler distal ends, for example, may be magnetized to optimize guidance for travel toward a ferromagnetic or magnetically attractive element integral or nearby a strut to be grasped.

In one embodiment of a grasp and lock mechanism, a loop or a strut or tubular structure of an anchor may be targeted by a bridging element. A bridging element may have a resting closed jaws position to a distal terminus that when activated after being delivered through the vasculature can be opened.

Once a strut is grasped by the jaws of a bridging element then the jaws are released and thereby will close and lock upon the target. The bridging element may then be cinched through a retro-chordae tendineae-anchor and locked in one embodiment.

In the case of grasping and locking onto a loop, in one embodiment a staple may be used. Inverse directional applications of these loops and bridging mechanisms may apply equally well.

A retro-chordae tendineae-anchor designed as a non-bulky flattened loop or mesh, button or rod may be disposed in at least one linear direction. It may be deployed such that a bridging element may join with it to form a T-shape. An anchor and bridge may be either pre-formed as a unit or in a second step a bridging element may be subsequently attached to a retro-chordae tendineae-anchor.

Once a bridge is pulled to an optimal tension as determined by observation of the magnitude of mitral regurgitation amelioration that is achieved by using an imaging technique such as echocardiography on a beating heart, a controlling cinch can be locked onto the bridging element. At that point the bridge element extending proximal to a cinch may be snapped, twisted or cut off and removed.

In anterior leaflet retro-chordae tendineae-anchor to great coronary vein anchor repairs a bridging element may be used to fully pull a retro-chordae tendineae-anchor and opposing atrial anchor completely together to essentially create an anchor to anchor bond. In such a bonded bridge arrangement leaflet tissue is bonded directly to atrial tissue. In the latter case there is no bridge implant material exposed to the blood stream. Only a tissue to tissue bridge is exposed to the blood stream. This achieves a roof-like covering of a posterior valve leaflet by an anchored anterior leaflet and by anchored atrial tissue advanced over a non-anchored posterior leaflet.

Once deployed a linearly formed retro-chordae tendineae-anchor, in one configuration, requires a bridging element that is generally attached at about the mid point of the retro-chordae tendineae-anchor facing an opposing anchor. The bridging element whether it is integrally constructed or not with the retro-chordae tendineae-anchor, is entered or exited to attach to the retro-chordae tendineae-anchor entering or exiting between the chords at about the midpoint of a leading edge of a leaflet in one embodiment. In this embodiment bridging to an opposing anchor is usually meant to occur at or about the midpoint of a great coronary vein anchor corresponding to a midpoint of the posterior mitral leaflet and corresponding annulus.

One mechanism to lock and cinch a bridge from an anterior leaflet retro-chordae tendineae-anchor to an opposing atrial related anchor is to pass an attached retro-chordae tendineae-anchor bridge through a loop extending from a great coronary vein anchor or from a suspended anchor or a from a right atrial or vena cava related anchor to enable an integral bridge to grasp and lock onto the loop.

Or a bridge attached to one anchor may slide through a loop of another bridge attached to another anchor and then release an expanded member larger than the opposing loop so that when a bridge with an expandable member the member of which is then expanded and pulled back it cannot slip through the loop. This mechanism acts as an anchor between two bridges as this anchor does not anchor into tissue. Such interlocking bridging element systems may be used to create a bridge that locks onto an opposing bridge instead of onto another anchor.

Another attachment mechanism of a retro-chordae tendineae-anchor to opposing anchor is accomplished using a non-adjustable but pre-determined bridge length mechanism. In one such application a staple-like feature is attached to a strut of a retro-chordae tendineae-anchor. In this application a staple-like terminus of a bridge length from an engagement end of a staple-like device for a retro-chordae tendineae-anchor bridging element is designed for engagement to an opposing anchor. The bridging element is pre-determined to be of fixed length and would require no truncation after cinching. The staple-like device extends in an overlapping fashion beyond a proximate edge of an opposing anchor attachment point. By means of a delivery catheter or tool a staple is advanced toward an opposing anchor attachment mechanism, for example a strut. Upon encountering an opposing anchor strut the staple is closed. Although this example of a bridge staple on strut instead of bridge staple to bridge loop may be used in an adjustable cinch application, it may also be applied as in this embodiment where no further cinching, adjustment, or proximal bridge disconnection steps are needed. Other bridge locking mechanisms may be applied in non-adjustable length bridging methods as well.

In other embodiments at least two bridging elements exiting or entering at different points between chords may be attached to a retro-chordae tendineae-anchor with the other ends of these bridging elements attached to an opposing anchor or joined to each other prior to attaching to another anchor.

A retro-chordae tendineae-anchor bridging element may be integrally constructed or may be independently attached during implantation. From there a retro-chordae tendineae-anchor bridging element may be attached to an opposing anchor directly or to another bridging element that is attachable or integral to an opposing anchor. These combinations may be applied to achieve either one or any combination of leaflet anchored fixation, opposing leaflet roofing or annulus stabilization or shortening.

A bridging element may also be attached integrally or independently to or from a suspension-scaffold anchor deployed above or near the valve annulus. Leaflet anchoring, opposing leaflet roofing or annulus stabilization or shortening could also be achieved through bridging to a retro-chordae tendineae-anchor or a great coronary vein.

A novel central bridging element to link at least one intra-cardiac or intra-vascular anchor to another may be integrally formed to have jaws on its distal terminus. It may also have at least one one-way compressible and expandable chevron brace on its body. It may also have a proximal loop for engagement with a proximal pulling instrument. All these features may be integrally applied into the same element eliminating any joints or fixtures. A shape memory alloy or possibly an elastic alloy or a polymer may be used to construct this element.

### IV. Methods of Delivery

Standard imaging techniques of fluoroscopy, angiography, echocardiography, ultrasound and advanced techniques of magnetic resonance imaging may be used to deliver and implant these devices properly surgically or through the vasculature.

Methods of retro-chordae tendineae-anchor delivery through the vasculature into a left atrial position include, but are not limited to trans-arterial and trans-septal approaches. The other anchor and bridging components of the systems implanted may be delivered by the same or different trans-septal or trans-arterial routes or into the vena cava and right atrium by a transvenous route.

In one trans-arterial retro-chordae tendineae-anchor delivery approach a delivery catheter may be advanced from a peripheral artery through a sheath, retrograde through the aortic valve. Once inside a left ventricle, a catheter would retroflex in the area between the papillary muscles. The catheter is then to exit from behind the chords of one of the leaflets into the front of the chords into the central orifice of the mitral valve blood flow pathway. From here it may ascend upstream between the leaflets for access to any left atrial structure.

In a case of anchoring an anterior mitral leaflet, once a catheter is passed through the chords, al retro-chordae tendineae-anchor bridging element may be advanced and attached to a posterior anchor or to a suspension-scaffold anchor or to a right atrial or a vena cava anchor across the atrial septum or a bridge extending from any of these opposing anchors.

In the case of a posterior anchor in a great coronary vein being attached to by an anterior leaflet anchor, a delivery catheter advanced from beneath an anterior leaflet would ride on an upstream surface of a posterior leaflet as it courses toward an anchor located in a great coronary vein. Some methods for linking great coronary vein anchors across an atrial wall into a left atrial chamber are described in our prior patent filing.

A retro-chordae tendineae-anchor may be pushed out of a delivery catheter proximate its terminal distal end into a space behind the chordae tendineae of a leaflet of a mitral valve, relative to the central flow through the mitral orifice. A retro-chordae tendineae-anchor may be an inflatable balloon-like structure, or an expandable flattened or three dimensional mesh or matrix that is of sufficient size when expanded, and of sufficient strength and construction so as not to rupture or herniate through the chords when the retro-chordae tendineae-anchor is pulled on at least at one single point.

Retro-chordae tendineae-anchor pulling forces may originate at any point of bridging that is anchored outside the space confining a retro-chordae tendineae-anchor. A bridge may attach to a retro-chordae tendineae-anchor at any point within or on any surface of a retro-chordae tendineae-anchor.

In one trans-septal approach to deliver a retro-chordae tendineae-anchor first a peripheral vein is entered and a sheath is advanced to the right atrium. The fossa ovalis of the atrial septum is approached with a hollow needle which is passed across the atrial septum. A guide wire is passed through the needle which is withdrawn. A guide catheter is passed into the left atrium.

Through a trans-septal guide catheter a retro-chordae tendineae-anchor delivery catheter may be advanced through a bridge loop extending from an anchor previously placed in a great coronary vein, a right atrium, a vena cava or a suspension-scaffold anchor. A retro-chordae tendineae-anchor catheter is then passed into the mitral valve orifice, flexed and passed behind the chords. There a retro-chordae tendineae-anchor is deployed and expanded into place. A retro-chordae tendineae-anchor delivery catheter is withdrawn and a retro-chordae tendineae-anchor bridge is extended back through a bridge loop from an opposing anchor. A retro-chordae tendineae-anchor bridge may then be cinched down on an opposing anchor loop with the excess bridging element being freed by previously described means and removed.

A method is described of treating atrioventricular valve regurgitation using radiographic, and ultrasonic or comparable imaging.

First use a transvenous catheter passed into a great coronary vein to deploy along a majority portion of its length a single tubular solid or hollow longitudinal member or a vascular stent like anchor with at least one longitudinal member.

Each member is made nearly parallel to a length of the vessel and oriented toward the endocardium. Then advance an outer containment-catheter of an anchor-bridge-anchor kit through a vasculature into a left heart chamber by a right to left atrial standard trans-septal technique or through a retrograde trans-arterial and trans-aortic valve route.

Then advance a catheter anchor-bridge-anchor kit's components as an assembled unit or advance its individual components or partially assembled components sequentially. Direct a containment-catheter to a point approximately near a midpoint of posterior mitral valve leaflet onto an endocardial surface of a great coronary vein.

Advance a central bridging element through a containment-catheter and beyond its distal aperture to allow expansion of a central bridging element's distal jaws onto a longitudinal member of an anchor in a great coronary vein.

Advance a pushing-catheter within a containment-catheter to engage a proximal aspect of a cylinder-like sleeve covering a central bridging element so that the sleeve advances distally to engage a seating point on the jaws of the proximal aspect of the central bridging element distally located jaws thereby advancing the jaws distally and locking the jaws onto a longitudinal anchor member that is within and parallel to a great coronary vein.

Withdraw a containment-catheter proximally enough at least to allow a proximal anchor to expand against tissue that has been transgressed by a catheter system and selected as a proximal anchor site.

Hold a slotted pushing-catheter in place on a proximal aspect of a sleeve upon which a proximal anchor is mounted.

Pull a pulling-catheter upon a proximal loop of a central bridging element, a pulling catheter being within a slotted pushing-catheter which is being held against a sleeve over a central bridging element.

Shorten the distance between a proximal and a distal anchor.

Pull a distal anchor proximally preventing back slippage of a central bridging element in a proximal direction by its jaws being distally anchored on a distal anchor.

Prevent distal slippage of a central bridging element by chevron braces mounted on it that are expanded sequentially a corresponding slot of a pushing catheter as a central bridging element is pulled proximally through a proximal aperture of a sleeve which is just outside a central bridging element.

Use ultrasonic or comparable imaging means to assess mitral regurgitation improvement for optimal anchor-bridge-anchor length adjustment.

Releasing a pulling instrument's jaws from a proximal loop of a central bridging element; withdraw a pushing and a pulling catheter instruments and an outer containment catheter from a vasculature.

Another method of treating atrioventricular valve regurgitation uses a catheter for advancing through a transvenous route with a magnet at its distal end into the great coronary vein a stent-like device. The magnet is then centered near a midpoint of the posterior mitral valve annulus. This is used to better guide a containment catheter with a magnet proximate its distal tip or a ferromagnetic or magnetic set of jaws on a central bridging element toward a great coronary vein stent-like device through the left atrium.

The magnet catheter is removed from the great coronary vein after the jaws of the central bridging element are secured on the strut of the stent-like device.

The methods, devices and systems developed for the mitral valve, may be adapted to be applied to the tricuspid atrioventricular valve as well.

### V. Infra-Leaflet Buttresses

In one embodiment, an infra-leaflet-buttress can be expanded and confined beneath a posterior mitral leaflet to advance the leaflet into a more upstream and central, fixed position to achieve leaflet closure to a determinable extent.

In a posterior leaflet embodiment, an infra-leaflet-buttress can be delivered behind the chords of a posterior mitral leaflet relative to the central flow through the mitral valve orifice. In this case, an infra-leaflet-buttress is fully expanded in all directions and thereby confined by the structures of the ventricular wall posteriorly and the papillary muscles inferiorly, the leaflet superiorly and the chords anteriorly and laterally.

In securing an infra-leaflet-buttress in place beneath the posterior mitral leaflet, the space in which the infra-leaflet-buttress is confined has no areas upon which it cannot rest. The circumferential support structure contact provided in the case of the posterior mitral leaflet is all that may be required to firmly secure the retro-chordae tendineae-anchor in the beating heart.

Combination repairs may include, for example, an isolated posterior infra-leaflet-buttress repair applied in conjunction with any anchor to anchor repair, e.g., with a retro-chordal anchor previously described.

An infra-leaflet-buttress may be deployed by a catheter being placed between the chords and an infra-leaflet-buttress being extruded from the terminus of the catheter and expanded behind the chords of the leaflet which may alone be sufficient to fix the infra-leaflet-buttress in place. Attachment by a separate stapling mechanism or another type of attachment mechanism attaching an infra-leaflet-buttress to at least one chord or leaflet edge may optionally be completed before the catheter is released from the infra-leaflet-buttress.

### VI. Illustrative Embodiments

The accompany drawings show illustrative embodiments of the technical features described above.

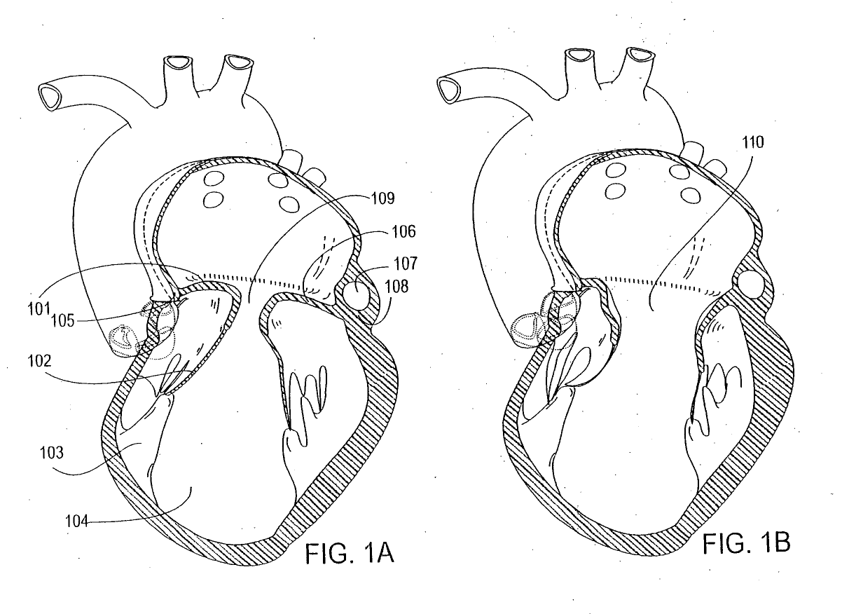

FIG. 1a: Shown in FIG. 1a is a sagittal view of the left heart through the middle of the anterior leaflet 101 and posterior leaflet 106 of the mitral valve. This view is the dilated heart 104 in systole when the leaflets should be closed but cannot meet 109 fully because of a dilated heart and annulus 108 causing functional mitral regurgitation. The anterior annulus 105 is widened from the posterior annulus 108. The chordae tendineae 102 and the papillary muscles 103 are displaced downward and outward thus tensioning the leaflets during systole preventing proper coaptation in systole. The great coronary vein 107 is shown in the left atrial wall above the mitral annulus.

FIG. 1b: Shown is a sagittal view of the left heart through the middle of the anterior and posterior leaflets of the mitral valve. This view is the heart in diastole when the leaflets open 110 as shown in a dilated heart and annulus condition associated with functional mitral regurgitation.

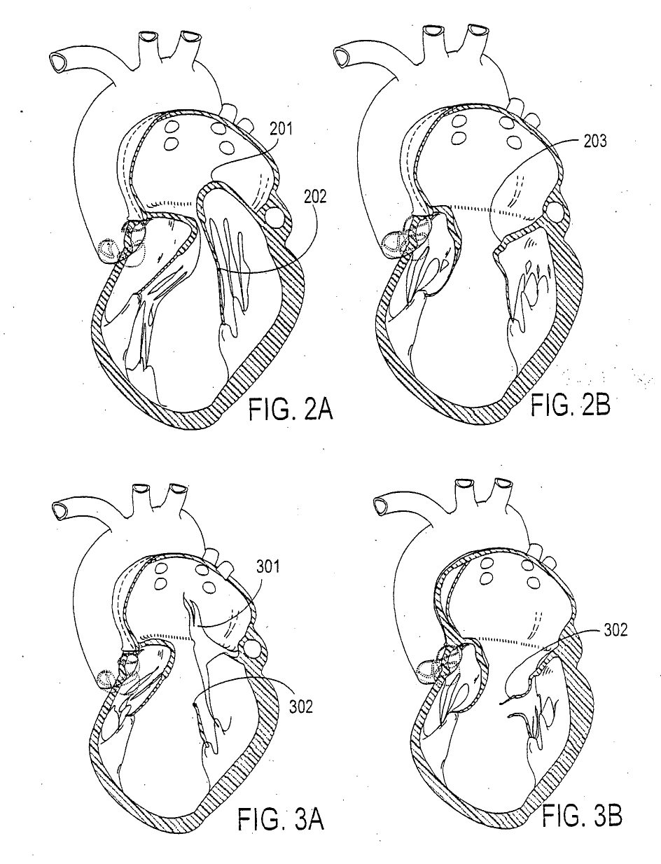

FIG. 2a: Shown is a sagittal view of the left heart through the middle of the anterior and posterior leaflets of the mitral valve. This view is the heart in systole with a prolapse condition of the posterior leaflet 201 of the mitral valve leading to organic or synonymously structural mitral regurgitation. The chordae tendineae 202 of the posterior leaflet are also elongated. Either or both leaflets may have a prolapsing condition. The prolapsing leaflet is floppy with excessive tissue and during systole is able to rise above it normal closure point near the annulus. When the prolapsing leaflet rises above the annulus it diverges from coaptation with the opposing leaflet resulting in mitral regurgitation.

FIG. 2b: Shown is a sagittal view of the left heart through the middle of the anterior and posterior leaflets of the mitral valve. This view is the heart in diastole with a floppy prolapse condition of the posterior leaflet 203 of the mitral valve that can lead to organic or synonymously structural mitral regurgitation. In this view the floppiness or excessive looseness of the posterior leaflet throughout its structure is seen by the manner in which the chordae and leaflet hang into the ventricle compared to a normal anterior leaflet.

FIG. 3a: Shown is a sagittal view of the left heart through the middle of the anterior and posterior 301 leaflets of the mitral valve. This view is the heart in systole with a ruptured chordae tendineae 302 condition of the middle scallop of the posterior leaflet of the mitral valve leading to a flail leaflet causing organic or structural mitral regurgitation.

FIG. 3b: Shown is a sagittal view of the left heart through the middle of the anterior and posterior leaflets of the mitral valve. This view is the heart in diastole with a ruptured chordae tendineae 302 condition of the middle scallop of the posterior leaflet of the mitral valve associated with a flail leaflet causing organic or structural mitral regurgitation.

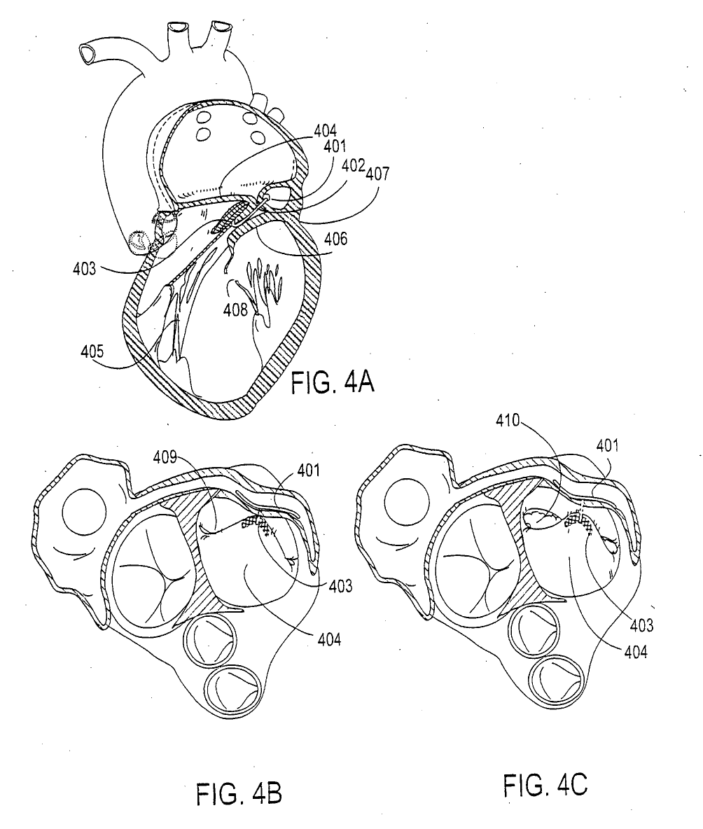

FIG. 4a: Shown a sagittal view the heart through the mid anterior and posterior mitral leaflet with embodiments of a tubular implant located in a great coronary vein 401 that is sized and configured to be bonded by a bridging element 402 extended to a retro-chordae tendineae-anchor 403 located behind the chordae tendineae of an anterior mitral leaflet at their junction with the leaflet bringing the leading edge of the leaflet across the left atrium in generally an anterior-to-posterior direction and bonding it to the leading edge of the posterior atrial wall tissue. The anterior mitral valve leaflet 404 is advanced more than could naturally be accomplished into a more upstream and central position for a given heart. The anterior leaflet meets the posterior atrial wall tissue leading the great coronary vein anchor 402 toward it. The joining of the posterior atrial wall and the anterior leaflet in varying proportion creates a roof for the posterior mitral leaflet 406 against which to close during systole. In addition the anchor to anchor bonding advances the posterior mitral annulus 407 anteriorly especially significantly in cases of functional mitral regurgitation when annulus dilation in the anterior to position or septal to lateral dimension is etiologic. The posterior mitral valve leaflet with structural causes for regurgitation may also be treated by this system. Such a lesion shown as a ruptured posterior chordae tendineae 408 leading to a flail posterior mitral leaflet may be treated by this device system.

FIG. 4b: Shown is a transverse section through the top of the atrioventricular heart valves shown in the systolic phase of the cardiac cycle. A great coronary vein tubular anchor 401 is seen being pulled anteriorly with the posterior wall of the left atrium by being bonded to a retro-chordae tendineae-anchor 403 located behind the chordae tendineae of the anterior leaflet 404 of the mitral valve. The leading coaptation edge of the posterior mitral vale leaflet 409 is seen meeting the anterior mitral valve leaflet in systole to create an effective closure to control mitral regurgitation.

FIG. 4c: Shown is FIG. 4c which identical to FIG. 4b except that this heart is shown in diastole such that the leading coaptation edge of the posterior mitral valve leaflet 410 is opened away from the anterior mitral valve leaflet to allow antegrade blood flow during ventricular diastole.

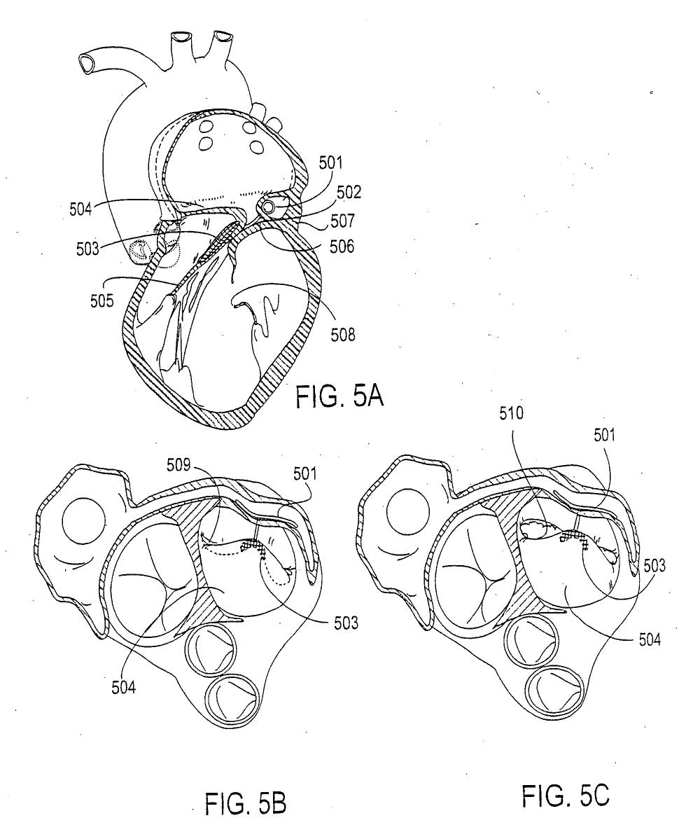

FIG. 5a: Shown is a sagittal view of the left heart through the middle of the anterior and posterior leaflets of the mitral valve. Shown is an anterior leaflet 504 beneath which is a retro-chordae tendineae-anchor 503 bridged with a bridging element 502 to a posterior atrial wall anchor 501 located inside the great coronary vein. The retro-chordae tendineae-anchor is placed behind the chordae tendineae 505 of the leaflet relative to the central valve orifice. The anterior leaflet is fixed into a more upstream and central or posterior position than was naturally possible for a given heart without the anchor system. This system may treat mitral regurgitation due to a dilated annulus, or a prolapse or a ruptured chord condition of either the anterior or posterior valve leaflet. In this view the posterior mitral leaflet 506 has become partially roofed by the anterior mitral leaflet and the posterior atrial wall. The posterior mitral annulus 507 has also been moved more anteriorly. The posterior mitral leaflet is shown closing fully in ventricular systole against the roof created by the anchor to anchor system above it and despite the ruptured chordae tendineae 508 that would otherwise lead to a flail mitral leaflet.

FIG. 5b: Shown is a transverse view through the top of the atrioventricular valves shown in systole.

Shown is an anterior mitral leaflet 504 beneath which is a retro-chordae tendineae-anchor 503 bridged to a posterior atrial wall anchor 501 located inside the great coronary vein. The anterior leaflet is fixed into a more central or posterior position than was naturally possible without the anchor system. This system may treat mitral regurgitation due to any cause including dilated annulus, or a prolapse or a ruptured chord condition of either the anterior or posterior valve leaflet. In this view the leading edge of the posterior mitral leaflet 509 has become partially roofed by the anterior mitral leaflet and the posterior atrial wall. The posterior mitral annulus has also been moved more anteriorly. The posterior mitral leaflet is shown closing fully in ventricular systole 509.

FIG. 5c: Shown is FIG. 5c which is identical to FIG. 5b except that the heart is in the diastolic cycle so that the leading edge posterior leaflet of the mitral valve 509 has opened or fallen away from the anterior leaflet to allow antegrade blood flow.

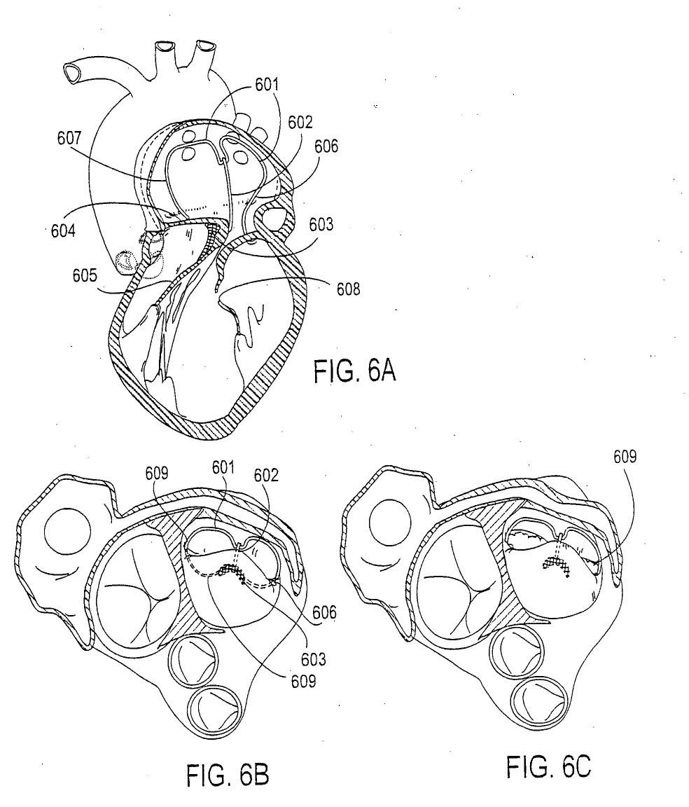

FIG. 6a: Shown is a sagittal view of the left heart through the middle of the anterior and posterior leaflets of the mitral valve. Shown is a suspension-scaffold 601 with a bridging element 602 to a retro-chordae tendineae-anchor 603 of the anterior mitral leaflet 604. The anterior leaflet chordae tendineae 605 are tensioned so that the anterior leaflet is advanced more than is naturally possible into a more upstream and central position for a given heart. The suspension-scaffold 601 has two descending struts 606 and 607 that in this embodiment pass through each commissure to seat the scaffold by curving around the annulus under compression there and against the atrial wall. This system may treat mitral regurgitation due to a dilated annulus, or a prolapse or a ruptured chord condition of either the anterior or posterior valve leaflet 608. In this view the posterior mitral leaflet has become partially roofed by the anterior mitral leaflet. The posterior mitral leaflet is shown closing fully in ventricular systole.

FIG. 6b: Shown is a transverse view through the top of the atrioventricular valves shown in systole.

Shown is an anterior leaflet retro-chordae tendineae-anchor 603 that is bridged 602 to a suspension-scaffold 601 seated on the atrial wall anchor and that hugs the annulus at the commissures 606 and 607. The anterior leaflet is fixed into a more upstream and central or posterior position than was possible without the anchor system. This system may treat mitral regurgitation due to a dilated annulus, or a prolapse or a ruptured chord condition of either the anterior or posterior valve leaflet. In this view the posterior mitral leaflet leading coaptation edge 609 has become partially roofed by the anterior mitral leaflet. The posterior mitral leaflet is shown closing fully in ventricular systole.

FIG. 6c: Shown is FIG. 6c which is identical to FIG. 6b except that this transverse view through the top of the atrioventricular valves is shown in diastole. Therefore the posterior mitral leaflet leading coaptation edge is shown falling open in ventricular diastole 609.

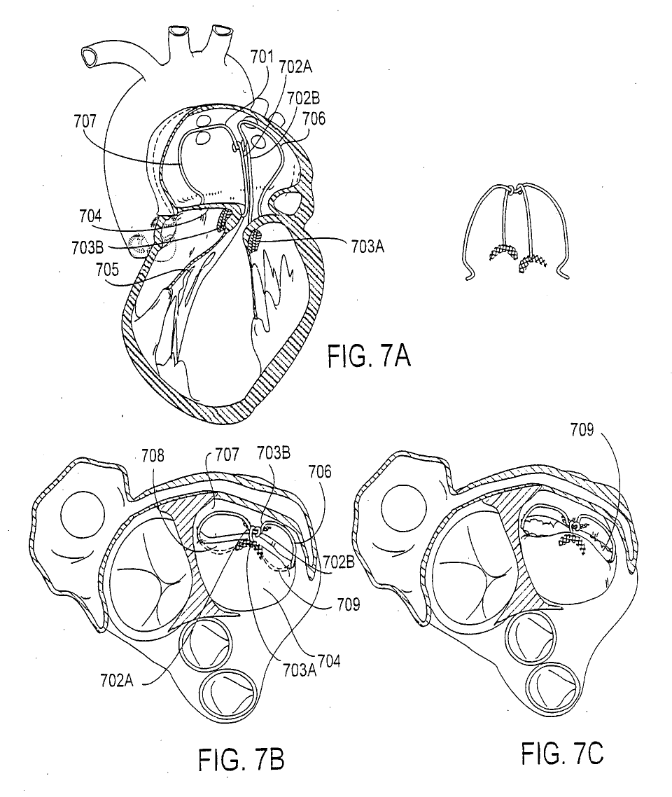

FIG. 7a: Shown is a sagittal view of the left heart through the middle of the anterior and posterior leaflets of the mitral valve. Shown is a suspension-scaffold 701 with a bridging element 702A to a retro-chordae tendineae-anchor 703A of the anterior mitral leaflet 704. The anterior leaflet chordae tendineae 705 are tensioned so that the anterior leaflet is advanced more than is naturally possible for a given heart into a more upstream and central position. The suspension-scaffold 701 has two descending struts 706 and 707 that in this embodiment pass through each commissure to seat the scaffold by curving around the annulus under compression there and against the atrial wall. This system may treat mitral regurgitation due to a dilated annulus, or a prolapse or a ruptured chord condition of either the anterior or posterior valve leaflet 608. In this view the posterior mitral leaflet has become partially roofed by the anterior mitral leaflet. The posterior mitral leaflet is shown closing fully in ventricular systole.

FIG. 7b: Shown is a transverse view through the top of the atrioventricular valves shown in systole.

Shown is an anterior leaflet retro-chordae tendineae-anchor 603 that is bridged 602 to a suspension-scaffold 601 seated on the atrial wall anchor and that hugs the annulus at the commissures 606 and 607. The anterior leaflet is fixed into a more upstream and central or posterior position than was possible without the anchor system. This system may treat mitral regurgitation due to a dilated annulus, or a prolapse or a ruptured chord condition of either the anterior or posterior valve leaflet. In this view the posterior mitral leaflet leading coaptation edge 609 has become partially roofed by the anterior mitral leaflet. The posterior mitral leaflet is shown closing fully in ventricular systole.

FIG. 7c: Shown is FIG. 7c which is identical to FIG. 7b except that this transverse view through the top of the atrioventricular valves is shown in diastole. Therefore the posterior mitral leaflet leading coaptation edge is shown falling open in ventricular diastole 609.

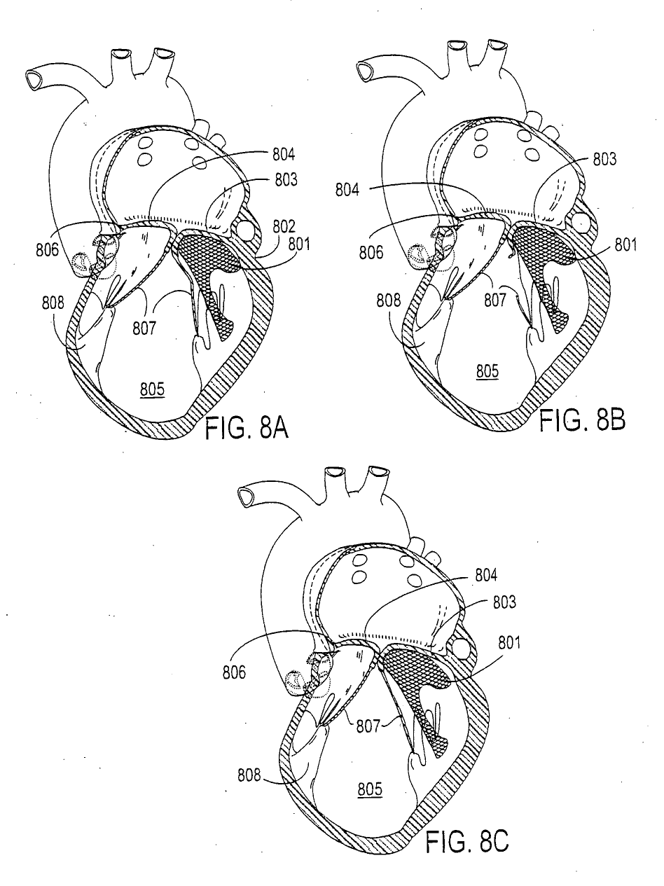

FIG. 8a: Shown in FIG. 8a is a sagittal view of the left heart through the middle of the anterior leaflet 804 and posterior leaflet 803 of the mitral valve. This view is the dilated heart in ventricular systole 805 when in an untreated heart the leaflets should be closed but cannot meet fully because of a dilated heart 805 and widening of the annulus between 802 and 806 causing functional mitral regurgitation. The chordae tendineae 807 and the papillary muscles 808 are displaced downward and outward thus tensioning the leaflets during systole preventing proper coaptation in systole. An infra-leaflet-buttress 801 is shown beneath the posterior mitral leaflet and behind the chordae tendineae. It is held up by the papillary muscles inferiorly and abuts the left ventricular wall posteriorly. It advances the posterior mitral leaflet 803 into a preferred position that is more upstream and centrally located than is naturally occurring for a given heart, toward the anterior leaflet 804 so that the more mobile anterior leaflet 804 may open away from the buttressed posterior leaflet 803 in ventricular diastole and close toward or come in contact with it during ventricular systole 805.

FIG. 8b: Shown in FIG. 8b is a sagittal view of the left heart through the middle of the anterior leaflet 804 and posterior leaflet 803 of the mitral valve. This view is the non-dilated heart in ventricular systole 805 when in an untreated heart the leaflets should be closed but cannot meet fully because of a flail posterior mitral leaflet 803 due to rupture of the chordae tendineae 807 from the papillary muscles 808. This prevents proper coaptation of the leaflets in systole. An infra-leaflet-buttress 801 is shown beneath the posterior mitral leaflet and behind the chordae tendineae. It is held up by the papillary muscles inferiorly and abuts the left ventricular wall posteriorly. It spreads tension across the entire leaflet and advances the posterior mitral leaflet 803 into a preferred position that is more upstream and centrally located than is naturally occurring for a given heart. This capability takes up any slack in the leaflet caused by the ruptured chord and fixes it in a position toward the anterior leaflet 804 so that the more mobile anterior leaflet 804 may open away from the buttressed posterior leaflet 803 in ventricular diastole and close toward it or come in contact with it during ventricular systole 805.

FIG. 8c: Shown in FIG. 8c is a sagittal view of the left heart through the middle of the anterior leaflet 804 and posterior leaflet 803 of the mitral valve. This view is the non-dilated heart in ventricular systole 805 when in an untreated heart the leaflets should be closed but cannot meet fully because of prolapse of a floppy posterior mitral leaflet 803 due to elongation of the leaflet tissue and the chordae tendineae 807 from the papillary muscles 808. This prevents proper coaptation of the leaflets in systole. An infra-leaflet-buttress 801 is shown beneath the posterior mitral leaflet and behind the chordae tendineae. It is held up by the papillary muscles inferiorly and abuts the left ventricular wall posteriorly. It spreads tension across the entire leaflet and advances the posterior mitral leaflet 803 into a preferred position that is more upstream and centrally located than is naturally occurring for a given heart. This capability takes up any slack in the leaflet caused by the elongation in the chordae tendineae and the leaflet tissue. It fixes the posterior leaflet in a position toward the anterior leaflet 804 so that the more mobile anterior leaflet 804 may open away from the buttressed posterior leaflet 803 in ventricular diastole and close toward it or come in contact with it during ventricular systole 805.

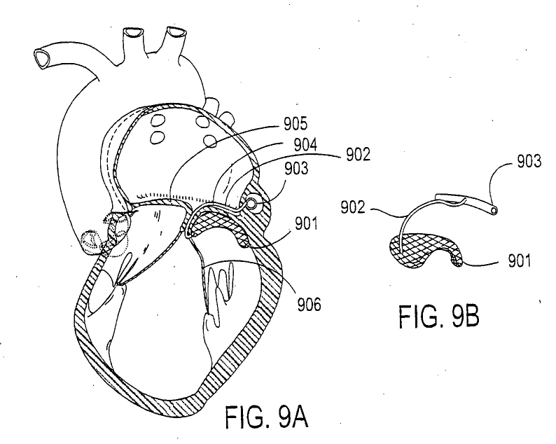

FIG. 9: Shown in FIG. 9a is a sagittal view of the heart through the middle of the anterior leaflet 905 and posterior leaflet 904 of the mitral valve. A retro-chordae tendineae-anchor 901 is placed beneath the posterior mitral leaflet behind the chordae tendineae 906. A bridging element 902 connects the retro-chordae tendineae-anchor to a tubular great coronary vein anchor 903. This anchor to anchor system also shown in free space in FIG. 9b may be used to advance a posterior mitral leaflet into a preferred position that is more upstream and centrally located than is naturally occurring for a given heart to treat mitral regurgitation due to dilated annulus or flail, prolapse and possible other structural lesions of the mitral valve.

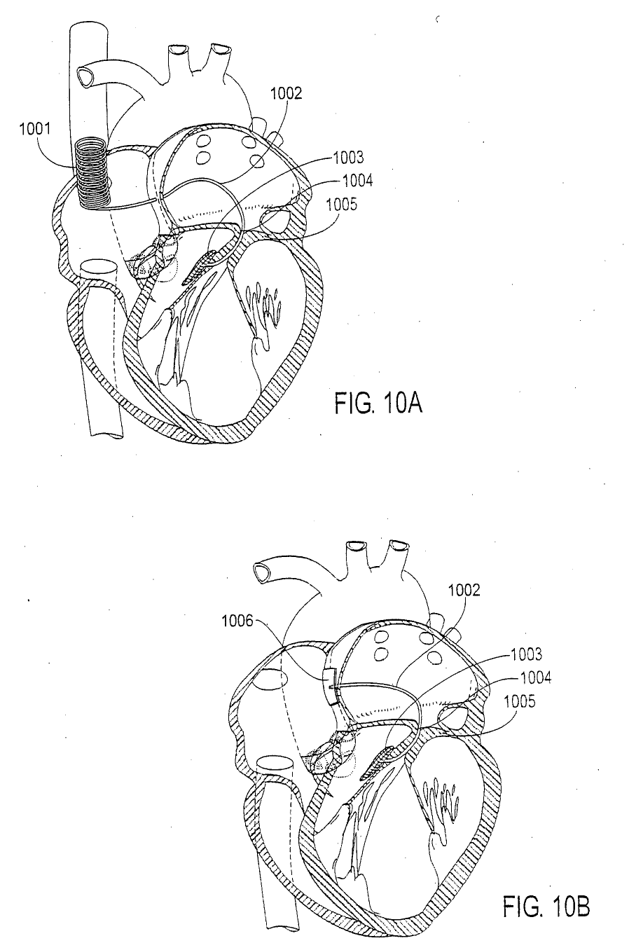

FIG. 10a: Shown in FIG. 10a is a sagittal view of the heart through the middle of the anterior leaflet 1004 and posterior leaflet 1005 of the mitral valve. A superior vena cava anchor 1001 is shown having a trans-septal bridging element 1002 that passes over the top of the anterior leaflet of the mitral valve 1004. There the bridging element connects to a retro-chordae tendineae-anchor 1003. This anchor to anchor system advances the anterior mitral leaflet into a more than natural possible upstream and central position to enhance it coaptation capability with the posterior mitral valve leaflet. This anchor to anchor system may be used to treat mitral regurgitation due to dilated annulus or flail, prolapse and other structural lesions of the mitral valve.

FIG. 10b: Shown in FIG. 10b is a sagittal view of the heart through the middle of the anterior leaflet 1004 and posterior leaflet 1005 of the mitral valve. A right septal anchor 1006 is shown having a trans-septal bridging element 1002 that passes over the top of the anterior leaflet of the mitral valve 1004. There the bridging element connects to a retro-chordae tendineae-anchor 1003. This anchor to anchor system advances the anterior mitral leaflet into a more than natural possible upstream and central position to enhance it coaptation capability with the posterior mitral valve leaflet. This anchor to anchor system may be used to treat mitral regurgitation due to dilated annulus or flail, prolapse and other structural lesions of the mitral valve.

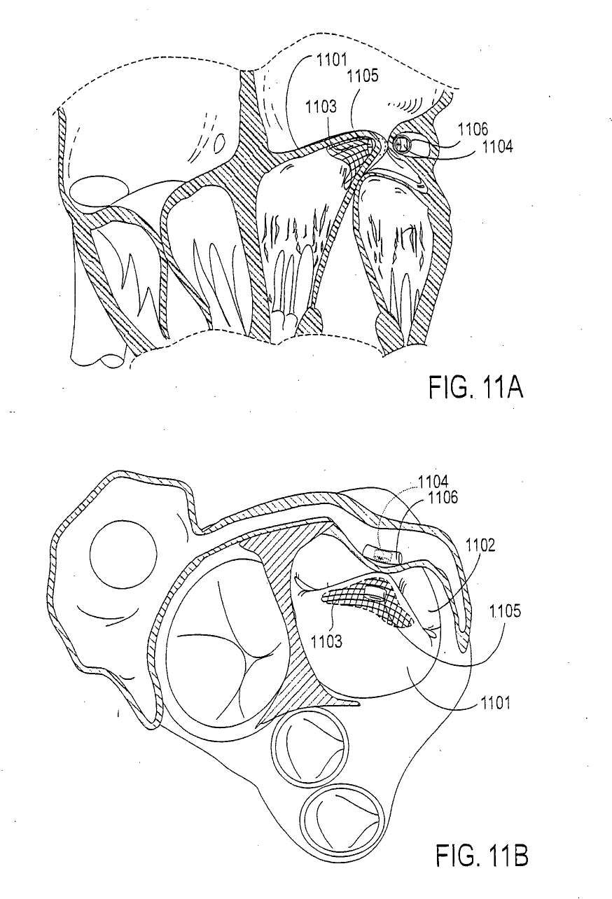

FIG. 11a: Shown in FIG. 11a is a sagittal view of the heart through the middle of the anterior leaflet 1101 and posterior leaflet 1002 of the mitral valve. A retro-chordae tendineae-anchor 1103 is placed beneath the anterior mitral leaflet 1101 and behind its chords. Within the retro-chordae tendineae-anchor 1103 is a magnet 1105 that is bonded to another magnet 1104. The latter magnet is within a tubular anchor 1106 residing within the great coronary vein. The anterior leaflet 1101 is advanced more than is natural possible into more upstream and central position where it is bonded to the posterior atrial wall. The posterior atrial wall has also been advanced forward by the force of magnetic attraction between the two anchors. The result is that the posterior leaflet of the mitral valve in some cases with favorable anatomy may be roofed completely. This may have application especially is etiologies of mitral regurgitation that are not associated with a dilated annulus.

FIG. 11b: Shown in FIG. 11b is a transverse view of the heart through the top of the atrioventricular valves shown in systole. Shown is the top of the anterior leaflet 1101 and posterior leaflet 1002 of the mitral valve. A retro-chordae tendineae-anchor 1103 is placed beneath the anterior mitral leaflet 1101 and behind its chords. Within the retro-chordae tendineae-anchor 1103 is a magnet 1105 that is bonded to another magnet 1104. The latter magnet is within a tubular anchor 1106 residing within the great coronary vein. The anterior leaflet 1101 is advanced into a preferred position that is more upstream and centrally located than is naturally occurring for a given heart where it is bonded to the posterior atrial wall. The posterior atrial wall has also been advanced forward by the force of magnetic attraction between the two anchors. The result is that the posterior leaflet of the mitral valve in some cases with favorable anatomy may be roofed completely. This would have application especially is etiologies of mitral regurgitation that are not associated with a dilated annulus.

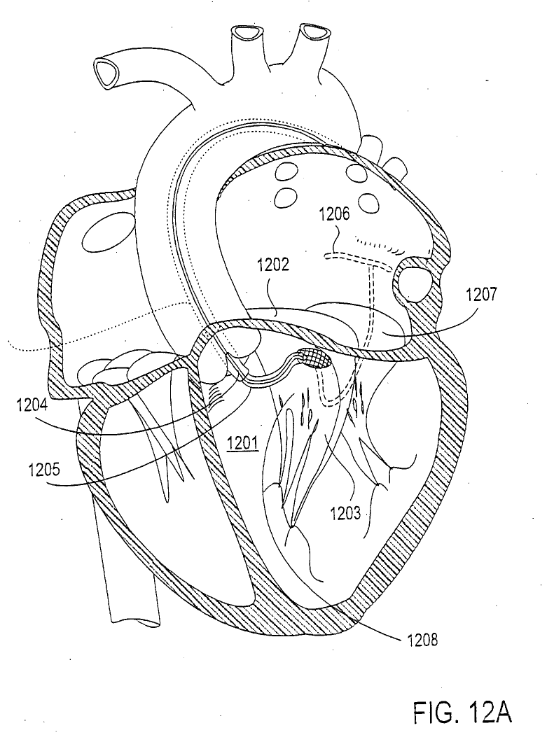

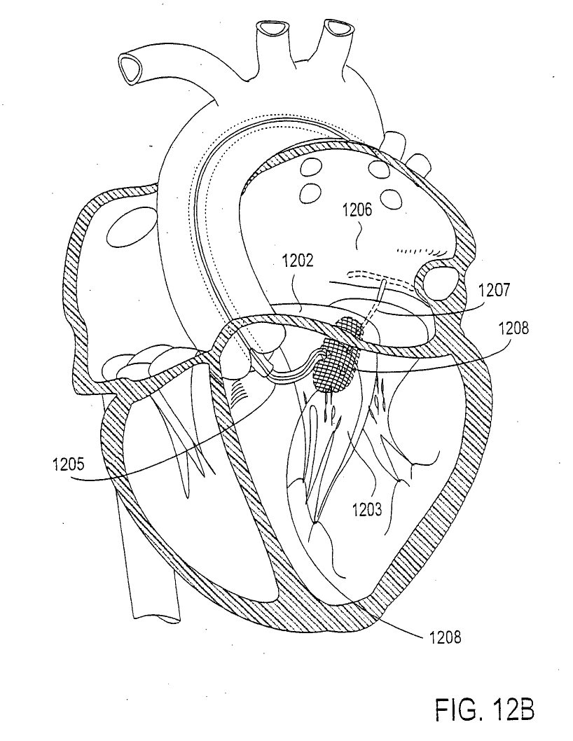

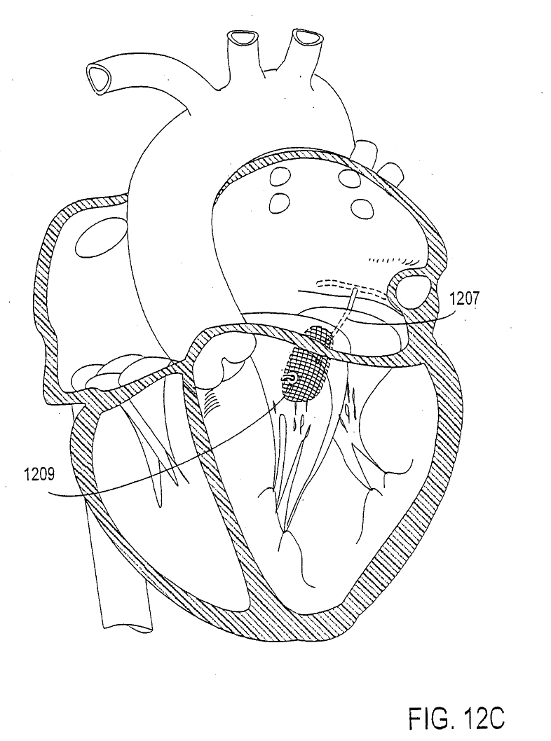

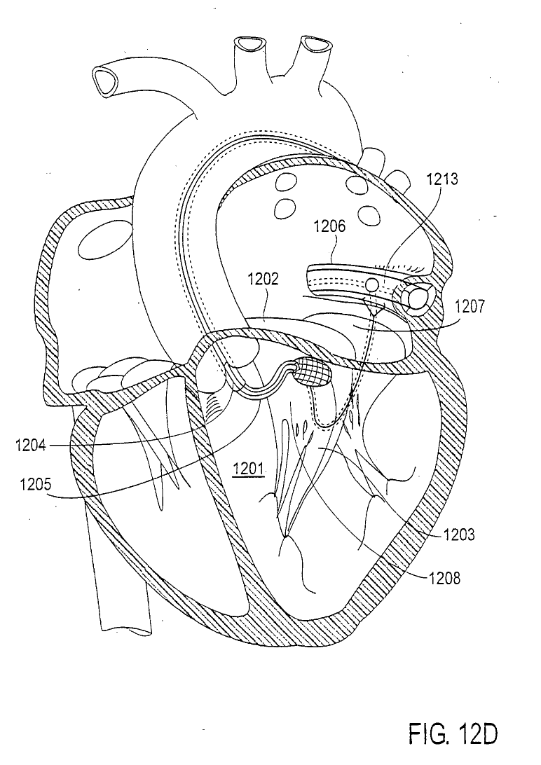

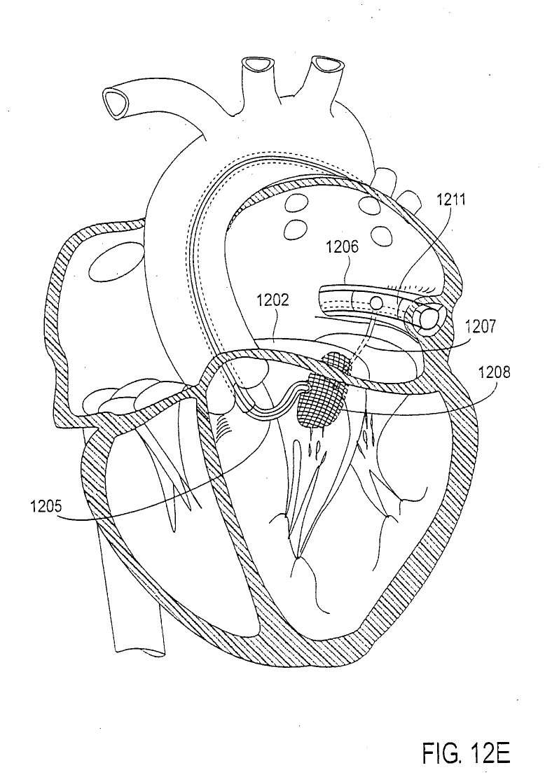

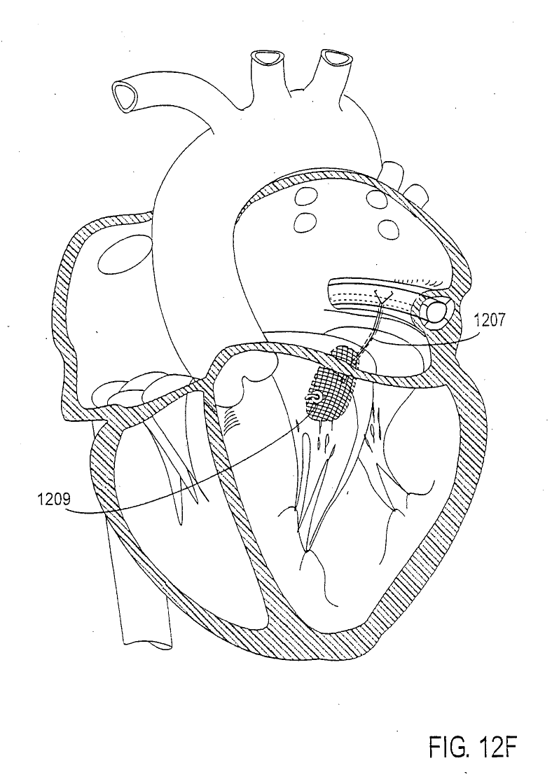

FIG. 12a: Shown in FIGS. 12a, b and c is a sagittal view of the heart through the left ventricular outflow tract 1201 anterior to the anterior leaflet 1202 of the mitral valve and its chordae tendineae 1203. Deployed inside a guiding catheter 1204 a catheter delivery system 1205 is shown in sequence passing through the vasculature. In FIG. 12a there is a T-shaped anchor 1206 that has been placed into the great coronary vein through the posterior atrial wall by techniques previously described using the delivery catheter shown. An integral bridging element 1207 is extending from the great coronary vein anchor 1206 back through the delivery catheter 1205. At the terminus of the delivery catheter is a retro-chordae tendineae-anchor 1208 through which the bridging element 1207 traverses. The delivery catheter 1205 and guide 1204 have been advanced from the femoral artery through the aortic valve into the left ventricular outflow tract 1201. Its terminus exited between the chordae tendineae 1203 of the anterior leaflet 1202 of the mitral valve to reach the posterior wall of the atrium for deploying the great coronary vein anchor 1206.

FIG. 12b: FIG. 12b is a sequential continuation of FIG. 12a. The delivery catheter 1205 has been pulled back just behind the chordae tendineae 1203 where the retro-chordae tendineae-anchor 1208 is being deployed. The bridging element 1207 is then pulled taught enough as to optimally as possible diminish mitral regurgitation.

FIG. 12c: FIG. 12c is a sequential continuation of FIG. 12b. Once the bridging element 1207 is adjusted to an appropriate length a cinch 1209 is advanced and locked on the bridge against the retro-chordae tendineae-anchor so that the anchor to anchor relationship is maintained.

FIG. 12d is a sagittal view of the heart through the left ventricular outflow tract anterior to the anterior leaflet of the mitral valve and its chordae tendineae. A delivery catheter is shown bringing a bridge and proximal anchor to link with a stent in the great coronary vein. A great coronary vein 1206 stent 1212 having a removable magnet 1213 is already in place having been placed through the os of the coronary sinus. Shown in this figure is a guide catheter 1204 with an inner implement catheter 1205 in which a retro-chordae-tendineae anchor 1206 is mounted on a fixed length central bridging element 1207 having distal terminal jaws 1211. This complex has been passed through the vasculature and through the aortic valve into the left atrium. As the bridge element is advanced the two fine jaws may be ferromagnetic in which case they may be guided by a magnet in a stent to the stent in the great coronary vein. This system may also be delivered through the atrial septum from the right atrium into the left atrium targeting the great coronary vein.

FIG. 12e is a continuation of the FIG. 12d delivery system now showing full attachment of the jaws 1211 of the bridging element 1207 onto a strut of the great coronary vein stent. The bridging element has been drawn taught back through the now deployed anterior mitral valve leaflet retro-chordae tendineae-anchor 1208. The implement catheter 1205 has set the bridging element onto the anterior aspect of the retro-chordae tendineae anchor and has controlled the length of the bridging element between the two anchors.

FIG. 12f is a continuation of the FIG. 12e delivery system showing final seating of the anchor 1203-bridge 1207-anchor 1212 system. The bridge element jaws 1211 are locked onto the stent 1212. The stent magnet 1213 has been removed. There is a lock 1209 on the proximal bridging element 1207. The implement catheter 1205 and guide sheath 1204 have been removed.

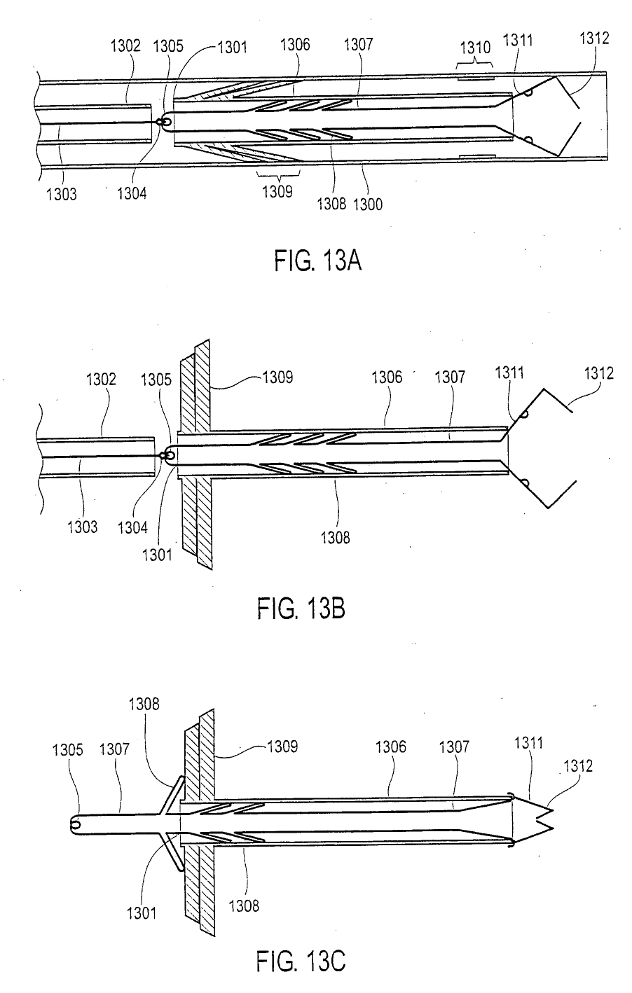

FIG. 13a is a schematic drawing of the initial stage pre-delivery phase application of a pre-delivery transvascular heart catheter system that may be applied in a right to left trans-atrial-septal approach or in a retrograde trans-arterial approach to the left heart chambers. It has an outer sheath 1300, a central bridging element 1307, a proximal anchor and control elements including a proximal bridge loop 1305 for linkage to a proximal pulling mechanism 1303 linked by controllable engagement mechanism like opening and closing jaws 1304. The outer delivery and containment sheath 1300 may have a magnet 1310 mounted near its distal tip to assist in locating another magnet located in a target elsewhere in the heart. 1302 is a proximal slotted push rod that can hold the bridge cylinder-like sleeve 1306 in place while the pulling mechanism 1303 pulls back the central bridge element 1307. An aperture 1301 in the cylinder-like 1306 allows the central bridge element 1307 to slide proximally out of the cylinder-like 1306. Chevron braces 1308 are mounted on the central bridge element 1307 and may open when the central bridge element is pulled far enough proximally through the aperture 1301. The chevron braces 1308 come to rest upon the proximal surface of the expanded proximal anchor 1309 which is adherently mounted at its center to the proximal end of the bridge cylinder-like sheath 1306. The chevron braces 1308 are compressible only in the proximal direction in which they are pointed and cannot collapse when a distally directed force is applied to central bridge element 1307. There is shown a delivery sheath or catheter magnet 1310 near the distal end of the outer delivery sheath or catheter 1300. At the distal terminus of the central bridging element is a pair of jaws 1312 held in a closed position by the outer sheath or catheter 1300. The jaws 1312 have recessed seating points 1311 that mate with the distal ends of the bridge cylinder-like sleeve 1306. The central bridging element 1307 is designed to slide within a bridge cylinder-like sleeve 1306. The central bridging element 1306 is held in place when the distal central bridging element jaws 1312 have locked onto an opposing anchor and the proximal chevron braces 1308 have expanded onto the proximal anchor 1309 after being cinched by the proximal pulling mechanism 1303.

FIG. 13b is a continuation of the next stage application of FIG. 13b where the outermost delivery containment sheath has been removed allowing the previously compressed distal jaws 1312 and proximal anchor 1309 to open and expand. The push rod catheter 1302 is slotted to allow the brace chevrons 1308 mounted on the central bridge element 1307 to open and brace the central bridging element from slipping distally against the expanded proximal anchor 1309. The central bridging element is pulled through the aperture 1301 in the bridge cylinder-like sleeve 1306 by a proximally located operator controlled pull wire 1303 reversibly linked by jaws 1304 to a proximal bridge loop 1305. A pulling instrument 1303 may also be useful in removal of a central bridging element 1307 after an indefinite period of time of implantation. The latter maneuver may be achieved by advancing an outer 1300 catheter over the central bridging element 1307 and placing a catheter with a pulling instrument 1303 with a jaws-like 1304 or a hook-like terminus to engage the proximal bridge loop 1305. The central bridge element 1307 may be pulled and rotated sufficiently to overcome its purchase on a distal anchor. The central bridging element 1307 is then pulled through a proximal anchor 1309 into the catheter 1300. If at least several weeks have passed since the system was placed this maneuver may leave the proximal anchor 1309 attached in place. If a proximal anchor then requires removal since it is collapsible standard transvascular grasping instruments and sleeve catheters may attach to and remove these anchors.

FIG. 13c is the continuation next stage application of FIG. 13b where the anchor-bridge-anchor system is fully deployed and locked with the proximal control elements removed. Shown is the central bridging element 1307 pulled proximally. This resulted in the bridge cylinder-like sleeve 1306 being pushed into the recessed seating points 1311 on the distal bridging element jaws 1312 thereby locking closed the jaws 1312 and fixing the central bridging element 1307 inside the bridge cylinder-like sleeve 1306 at the distal aspects of these elements. The proximal aspect of the bridge cylinder-like sleeve 1306 is also locked relative to the central bridging element 1307 by the chevron braces 1308 mounted thereon that have been pulled through the aperture 1301 in the bridge cylinder-like sleeve 1306.

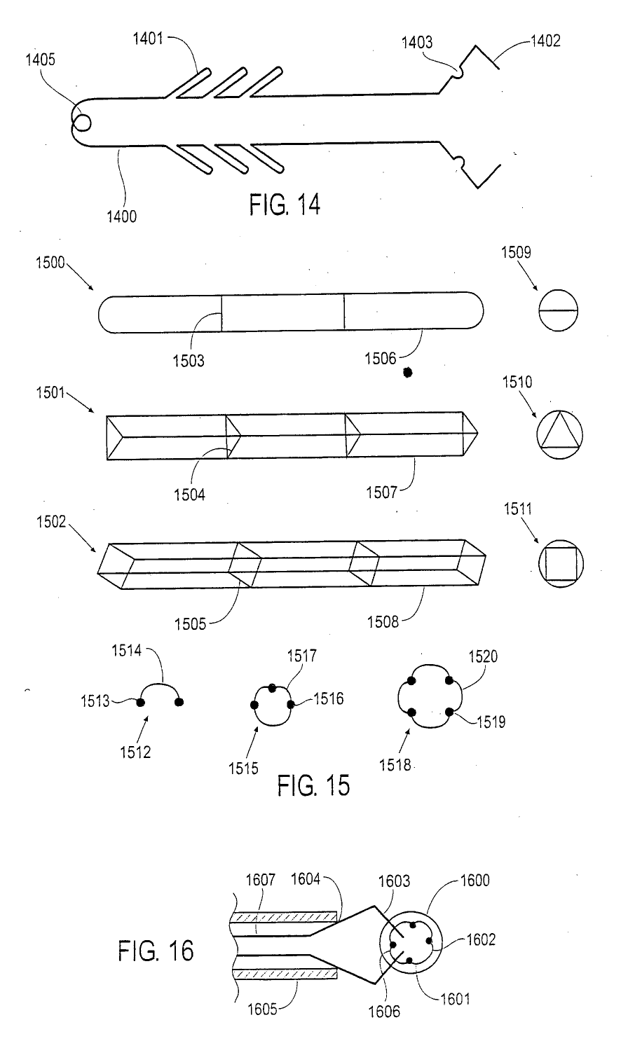

FIG. 14 is a schematic of an integrally formed single strand wire form central bridge element 1400 having integrally formed chevron braces 1401. The distal end has an integrally formed pair of jaws 1402 with recessed seating points 1403. The proximal end has an integrally formed proximal bridge loop 1405 for engaging a pulling instrument. A pulling instrument may pull a central bridge element through a proximal anchor for seating or central bridging element retrieval.

FIG. 15 is a schematic of three of the several possible embodiments claimed of great coronary vein large cell stent-like anchors 1500, 1501 and 1502 in longitudinal profile for attachment of bridging elements between two anchors. Transverse struts 1503, 1504 and 1505 may be perpendicular or at some other angle to the longitudinal struts 1506, 1507, and 1508 which may or may not be parallel to each other. Cross-sections of a great coronary vein with stent-like anchors having transverse struts crossing the vessel lumen are seen having two longitudinal struts 1509, three longitudinal struts 1510 and four longitudinal struts 1511. In cross-section again are three stent-like anchors 1512, 1515 and 1518 shown having curvilinear transverse struts 1514, 1517 and 1520 that conform to a vessel wall respectively having two 1513, three 1515 and four longitudinal struts 1519.

FIG. 16 is a schematic showing a cross-section of a great coronary vein 1600 within which is a four-longitudinal strut 1601 anchor having at least four transverse struts 1602. Jaws 1603 of a bridging element 1607 proximally attached to an opposing anchor, not shown, are locked onto an endocardial facing longitudinal strut 1606 of a great coronary vein anchor. A longitudinal sleeve 1605 on the bridging element 1607 has locked the jaws onto the strut 1606.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I/We claim:

1. An implant system to treat a regurgitant mitral heart valve comprising
a posterior anchor structure sized and configured to extend within a great cardiac vein along a posterior annulus of a mitral valve
an anteriorly anchoring infra-leaflet retro-chordae tendineae-anchor structure sized and configured to be engaged behind at least one chordae tendineae of a mitral valve leaflet,
at least one implant sized and configured to extend across a left atrium including a posterior anchoring region sized and configured to extend within an left atrium into a great cardiac vein and couple to a posterior anchor structure within a great cardiac vein, an anterior anchoring region sized and configured to extend from a more anterior retro-chordae tendineae-anchor, and
a bridging region between the posterior and anterior anchoring regions sized and configured to span a left atrium in a posterior-to-anterior direction, to hold in tension a bridging element between the posterior and anterior anchoring regions.

2. An implant system according to claim 1
wherein the anterior anchor structure is collapsible for placement within a catheter.

3. An implant system according to claim 1
wherein the retro-chordae tendineae-anchor anterior structure comprises a mesh-like structure or a balloon like structure, or a solid or hollow rod-like structure of any shape

4. An implant system according to claim 1
wherein the opposing anchoring structure for either the great coronary vein anchor or the retro-chordae tendineae-anchor is sized and configured for attachment on the interatrial septum, or at or near the fossa ovalis, or the superior vena cava, or the inferior vena cava.

5. An implant system according to claim 1
wherein the bridging region is sized and configured to extend in a posterior-to-anterior direction within the left atrium in an inferiorly path toward the mitral valve.

6. An implant system according to claim 1
wherein the bridging region comprises an elastic structure, or a wire-form structure, or a suture.

7. An implant system to treat a mitral heart valve comprising
an infra-leaflet-buttress designed to support a mitral valve leaflet into a preferred position that is more upstream and centrally located than is naturally occurring for a given heart, the buttress having a structure sized and configured to be large enough when placed or expanded to be held by the confines beneath a mitral valve leaflet behind the chordae tendineae, in front of the ventricular wall and above the papillary muscles.

8. A method treating atrioventricular valve regurgitation comprising using the implant system defined in claim 1.

9. A method treating atrioventricular valve regurgitation comprising using the implant system defined in claim 7.
